(19) United States
(12) Patent Application Publication
(10) Pub. No.: US 2023/0076952 A1
(43) Pub. Date: Mar. 9, 2023

(54) MULTI-FUNCTION SURGICAL INSTRUMENTS AND SELECTION ASSEMBLIES FOR MULTI-FUNCTION SURGICAL INSTRUMENTS

(71) Applicant: Covidien LP, Mansfield, MA (US)

(72) Inventors: Rajanikanth Mandula, Hyderabad (IN); Ravi Sekhar Gutti, Hyderabad (IN)

(21) Appl. No.: 17/825,021

(22) Filed: May 26, 2022

Related U.S. Application Data
(60) Provisional application No. 63/242,359, filed on Sep. 9, 2021.

(51) Int. Cl. A61B 18/14

(52) U.S. Cl. CPC A61B 18/1445; A61B 2018/1253

(57) ABSTRACT

A surgical instrument includes a housing, at least three deployable components selectively deployable relative to the housing from a storage to a use position, a deployment and retraction assembly, and a selection assembly. The deployment and retraction assembly includes an actuator, an output driver, and a gear assembly operably coupled therebetween such that actuation of the actuator drives the output driver. The selection assembly includes a coupling shaft movable between at least first, second, and third positions wherein the coupling shaft is operably coupled between the output driver and first, second, and third deployable components, respectively, of the at least three deployable components such that actuation of the actuator drives the output driver to deploy the respective first, second, or third deployable component.

### CROSS-REFERENCE TO RELATED APPLICATIONS

This application claims the benefit of, and priority to, U.S. Provisional Patent Application No. 63/242,359, filed on Sep. 9, 2021, the entire contents of which are hereby incorporated herein by reference.

### BACKGROUND

### Technical Field

The present disclosure relates to surgical instruments and, more particularly, to multi-function surgical instruments and selection assemblies therefor that facilitate selecting between different use configurations of the surgical instruments.

### Background of Related Art

Bipolar surgical instruments typically include two electrodes charged to different electric potentials to selectively apply energy to tissue. Bipolar electrosurgical forceps, for example, utilize both mechanical clamping action and electrical energy to control the heating of tissue to seal tissue. Once tissue is sealed or otherwise treated (e.g., cauterized, coagulated, desiccated, etc.), it is often desirable to cut the treated tissue. Accordingly, many forceps have been designed which incorporate a mechanical knife that effectively severs the tissue after tissue treatment, although electrical and electromechanical (dynamic or static) cutting may alternatively be utilized.

Monopolar surgical instruments, on the other hand, include an active electrode, and are used in conjunction with a remote return electrode, e.g., a return pad, to apply energy to tissue. Monopolar instruments have the ability to rapidly move through tissue and dissect through narrow tissue planes. Different electrode configurations may be utilized to achieve different tissue effects, to better access tissue to be treated, and/or for other purposes.

In some surgical procedures, it may be beneficial to use both bipolar and monopolar instrumentation, e.g., procedures where it is necessary to dissect through one or more layers of tissue in order to reach underlying tissue(s) to be treated, e.g., sealed. Further, it may be beneficial, particularly with respect to endoscopic surgical procedures, to provide a single instrument incorporating both bipolar and monopolar features, thereby obviating the need to alternatingly remove and insert the bipolar and monopolar instruments in favor of one another.

As can be appreciated, as additional functional components are added to a surgical instrument, additional deployment structures or deployment structures capable of actuating more than one component are required. However, multiple deployment structures and/or combined deployment structures may be limited by spatial constraints within the housing of the surgical instrument, functional constraints of the components (e.g., where a combined deployment structure imparts additional force requirements for deploying one or more of the components coupled thereto), and/or may overly complicate the operable components of the surgical instrument.

### SUMMARY

As used herein, the term “distal” refers to the portion that is being described which is further from an operator, e.g., a surgeon or a surgical robot, while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a surgical instrument including a housing, at least three deployable components, a deployment and retraction assembly, and a selection assembly. Each deployable component of the at least three deployable components is selectively deployable relative to the housing from a storage position to a use position. The deployment and retraction assembly includes an actuator, an output driver, and a gear assembly operably coupled between the actuator and the output driver such that actuation of the actuator drives the output driver. The selection assembly is operably coupled to the deployment and retraction assembly and includes a coupling shaft movable between at least a first position wherein the coupling shaft is operably coupled between the output driver and a first deployable component of the at least three deployable components such that actuation of the actuator drives the output driver to deploy the first deployable component, a second position wherein the coupling shaft is operably coupled between the output driver and a second deployable component of the at least three deployable components such that actuation of the actuator drives the output driver to deploy the second deployable component, and a third position wherein the coupling shaft is operably coupled between the output driver and a third deployable component of the at least three deployable components such that actuation of the actuator drives the output driver to deploy the third deployable component.

In an aspect of the present disclosure, the output driver is adapted to connect to a source of energy and the coupling shaft is configured to electrically couple the output driver to the first, second, and third deployable components in the respective first, second, and third positions of the coupling shaft. In such aspects, at least one of the first, second, or third deployable components may be configured as a monopolar electrode configured to supply monopolar energy to tissue in the use position thereof.

In another aspect of the present disclosure, the selection assembly includes at least second and third selectors extending from the housing. Each of the second and third selectors includes a driver at a free end thereof. Depression of the second and third selectors moves the coupling shaft, under urging by the corresponding driver, to the second and third positions, respectively.

In still another aspect of the present disclosure, the selection assembly includes a first selector extending from the housing. Depression of the first selector releases the coupling shaft for return from the second or third position thereof to the first position thereof.

In yet another aspect of the present disclosure, the selection assembly includes an input gear extending from the housing and an output gear coupled to the input within the housing. The output gear is operably coupled to the coupling shaft such that rotation of the input gear to first, second, and third orientations, respectively, moves the coupling shaft to the first, second, and third positions, respectively.

In another aspect of the present disclosure, rotatable actuation of the actuator of the deployment and retraction assembly drives the output driver of the deployment and retraction assembly to translate longitudinally. In such aspects, a first rotational actuation of the actuator in a first rotational direction translationally may drive the output driver in a first translational direction while and a second, subsequent rotational actuation of the actuator in the first rotational direction translationally drives the output driver in second, opposite translational direction.

In still yet another aspect of the present disclosure, the coupling shaft moves vertically between the first, second, and third positions. Alternatively, the coupling shaft moves along an arc between the first, second, and third positions.

Another surgical instrument provided in accordance with aspects of the present disclosure includes a housing, a shaft assembly extending distally from the housing, an end effector assembly disposed at a distal end of the shaft assembly, a plurality of deployable components, a deployment and retraction assembly, and a selection assembly. Each deployable component of the plurality of deployable components is selectively deployable relative to the end effector assembly from a storage position to a use position. The deployment and retraction assembly includes an actuator, an output driver, and a gear assembly operably coupled between the actuator and the output driver such that actuation of the actuator in a first manner drives the output driver in a second, different manner. The selection assembly is operably coupled to the deployment and retraction assembly and is transitionable between a plurality of different configurations to thereby operably couple the output driver of the deployment and retraction assembly to a different deployable component of the plurality of deployable components in each of the plurality of different configurations of the selection assembly. Actuation of the actuator drives the output driver to deploy the deployable component of the plurality of deployable components that is operably coupled to the output driver as selected based on the configuration of the selection assembly.

In an aspect of the present disclosure, the selection assembly includes a plurality of selectors extending from the housing. Depression of each selector of the plurality of selectors transitions the selection assembly to a different configuration of the plurality of different configurations.

In another aspect of the present disclosure, the selection assembly includes an input gear extending from the housing and rotatable between a plurality of different input gear orientations. Rotation of the input gear to each orientation of the different input gear orientations transitions the selection assembly to a different configuration of the plurality of different configurations.

In still another aspect of the present disclosure, the first manner of actuation of the actuator of the deployment and retraction assembly is a rotational actuation and the second manner of actuation of the output driver of the deployment and retraction assembly is a translational driving. In such aspects, a first rotational actuation of the actuator in a first rotational direction may translationally drive the output driver in a first translational direction while a second, subsequent rotational actuation of the actuator in the first rotational direction translationally drives the output driver in second, opposite translational direction.

In yet another aspect of the present disclosure, the selection assembly includes a coupling shaft operably coupled with the output driver. The coupling shaft is configured to move between a plurality of different positions. Each position of the plurality of different positions corresponds to one of the different configurations of the selection assembly. In each position of the plurality of different positions, the coupling shaft is operably coupled to a different deployable component of the plurality of deployable components.

In still yet another aspect of the present disclosure, the coupling shaft is movable in a first direction between the plurality of different positions and is movable in a second, perpendicular direction upon driving of the output driver to thereby deploy the corresponding deployable component of the plurality of deployable components. In such aspects, the selection assembly may include a plurality of selectors extending from the housing wherein depression of each selector of the plurality of selectors moves the coupling shaft to a different position of the plurality of different positions.

In another aspect of the present disclosure, the coupling shaft is movable along an arc between the plurality of different positions and is movable in a second direction perpendicular to a plane containing the arc upon driving of the output driver to thereby deploy the corresponding deployable component of the plurality of deployable components. In such aspects, the selection assembly may include a selection gear extending from the housing. Rotation of the selection gear moves the coupling shaft along the arc between the plurality of different positions.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a front, side, perspective view of a multi-function surgical instrument provided in accordance with the present disclosure in a retracted condition;

FIG. 2 is a rear, side, perspective view of the area of detail indicated as “2” in FIG. 1;

FIG. 3 is a side, perspective view of a proximal portion of the instrument of FIG. 1 with a portion of the housing removed to illustrate internal features therein;

FIG. 4A is a side view of a distal end portion of the instrument of FIG. 1 in a deployed condition wherein a first deployable component is deployed;

FIG. 4B is a side view of the distal end portion of the instrument of FIG. 1 in a deployed condition wherein a second deployable component is deployed;

FIG. 4C is a side view of a distal end portion of the instrument of FIG. 1 in a deployed condition wherein a third deployable component is deployed;

FIG. 5 is an enlarged, side, perspective view of a portion of the instrument of FIG. 1 as shown in FIG. 3, illustrating a selection assembly provided in accordance with the present disclosure and configured for use with the instrument of FIG. 1 to enable selection of a deployable component to be deployed, wherein the selection assembly is disposed in a first position selecting the first deployable component for deployment;

FIGS. 6A and 6B are side, perspective views illustrating the selection assembly of FIG. 3 in use with the instrument of FIG. 1 wherein the selection assembly is disposed in second and third positions, respectively, selecting the respective second and third deployable components for deployment;

FIG. 7A is a rear, perspective, partial transverse cross-sectional view of a proximal portion of the instrument of FIG. 1 illustrating another selection assembly provided in accordance with the present disclosure and configured for use with the instrument of FIG. 1 to enable selection of a deployable component to be deployed; and

FIG. 7B is an enlarged, rear, perspective view illustrating the selection assembly of FIG. 7A.

### DETAILED DESCRIPTION

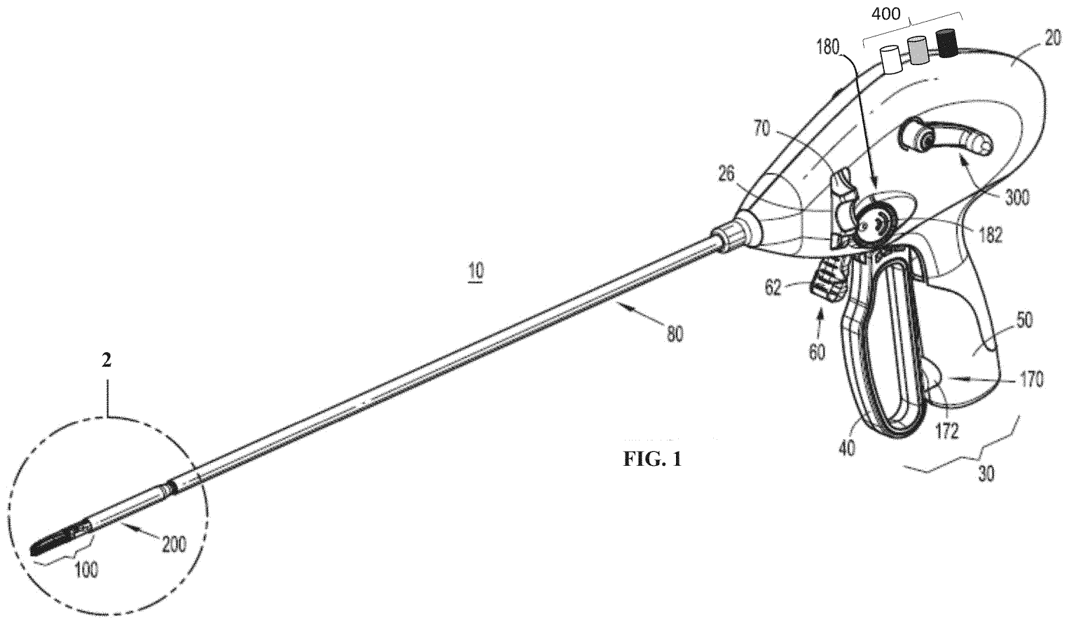

Referring generally to FIGS. 1-3, a multi-function surgical instrument provided in accordance with the present disclosure is shown generally identified by reference numeral 10. Instrument 10, as described below, is configured to operate in both a bipolar mode, e.g., for grasping, treating, and/or mechanically dissecting tissue, and a monopolar mode, e.g., for treating and/or electrically/electromechanically dissecting tissue. Further, with respect to the monopolar mode of operation, a selection assembly enables selection of one of a plurality of different monopolar electrodes to be deployed for use in the monopolar mode. Although the present disclosure is shown and described with respect to instrument 10 for use in bipolar and monopolar modes of operations with different selectable monopolar electrodes, the aspects and features of the present disclosure are equally applicable for use with any suitable multi-function surgical instrument having one or more modes of operation and a deployable component selected from a plurality of deployable components. Obviously, different connections and considerations apply to each particular instrument and the assemblies and/or components thereof; however, the aspects and features of the present disclosure remain generally consistent regardless of the particular instrument, assemblies, and/or components provided.

Instrument 10 generally includes a housing 20, a handle assembly 30, a trigger assembly 60, a rotating assembly 70, a shaft assembly 80, an end effector assembly 100, a drive assembly 140, a knife assembly 160, respective bipolar and monopolar activation assemblies 170, 180, a monopolar assembly 200, a deployment and retraction mechanism 300, and a selection assembly 400. Shaft assembly 80 (inner components of which are not shown) extends distally from housing 20 and supports end effector assembly 100 at a distal end thereof. Drive assembly 140 operably couples handle assembly 30 with end effector assembly 100 to enable selective manipulation of jaw members 110, 120 of end effector assembly 100. Knife assembly 160 is operably coupled with trigger assembly 60 to enable selective translation of a knife (not shown) of knife assembly 160 relative to end effector assembly 100. Deployment and retraction mechanism 300 is operably coupled with monopolar assembly 200 to enable selective deployment and retraction of monopolar assembly 200. Rotating assembly 70 enables selective rotation of end effector assembly 100 and monopolar assembly 200 relative to housing 20. Bipolar and monopolar activation assemblies 170, 180 enable the appropriate energy to be selectively delivered to end effector assembly 100 and monopolar assembly 200, respectively.

Instrument 10 may also include an electrosurgical cable (not shown) that connects instrument 10 to a generator (not shown) or other suitable power source, although instrument 10 may alternatively be configured as a battery-powered instrument having an on-board battery and generator (separate or combined with one another). The electrosurgical cable (not shown) includes wires (not shown) extending therethrough that have sufficient length to extend through housing 20 and/or shaft assembly 80 in order to provide energy to at least one of the electrically-conductive surfaces 112, 122 of jaw members 110, 120, respectively, of end effector assembly 100, e.g., upon activation of bipolar activation switch 172 of bipolar activation assembly 170 in the bipolar mode of operation. Similarly, one or more of the wires of the electrosurgical cable (not shown) extends through housing 20 and/or shaft assembly 80 in order to provide energy to monopolar assembly 200, e.g., upon activation of either of the monopolar activation switches 182 of monopolar activation assembly 180 in the monopolar mode of operation. As can be appreciated, additional wires (not shown) are provided to electrically couple the various inter-operable electrical components of instrument 10.

End effector assembly 100 is attached at the distal end of shaft assembly 80 and includes opposing jaw members 110, 120 pivotably coupled to one another. Jaw members 110, 120 are pivotably coupled to one another to permit movement of one or both of jaw members 110, 120 relative to the other between a spaced-apart position and an approximated position for grasping tissue between surfaces 112, 122. One or both of surfaces 112, 122 are adapted to connect to the source of energy (not shown), e.g., via one or more wires (not shown), and are configured to conduct energy through tissue grasped therebetween to treat tissue, e.g., cauterize, coagulate/desiccate, and/or seal tissue. More specifically, in aspects, end effector assembly 100 defines a bipolar configuration wherein surface 112 is charged to a first electrical potential and surface 122 is charged to a second, different electrical potential such that an electrical potential gradient is created for conducting energy between surfaces 112, 122 and through tissue grasped therebetween for treating tissue. Bipolar activation switch 172 of bipolar activation assembly 170 is operably coupled between the source of energy (not shown) and surfaces 112, 122 via one or more wires (not shown), thus allowing the user to selectively apply energy to surfaces 112, 122 of jaw members 110, 120, respectively, of end effector assembly 100 during a bipolar mode of operation.

Continuing with reference to FIGS. 1-3, end effector assembly 100 is designed as a unilateral assembly, i.e., where jaw member 120 is fixed relative to shaft assembly 80 and jaw member 110 is movable relative to shaft assembly 80 and fixed jaw member 120. However, end effector assembly 100 may alternatively be configured as a bilateral assembly, i.e., where both jaw member 110 and jaw member 120 are movable relative to one another and to shaft assembly 80. Further, in aspects, a longitudinally-extending knife channel (not shown) may be defined within one or both of jaw members 110, 120 to permit reciprocation of a knife (not shown) of knife assembly 160 therethrough, e.g., upon actuation of a trigger 62 of trigger assembly 60, to cut tissue grasped between jaw members 110, 120. Jaw members 110, 120 of end effector assembly 100 may otherwise be configured similar to those of the end effector assembly detailed in U.S. Pat. No. 9,655,673, the entire contents of which are hereby incorporated herein by reference.

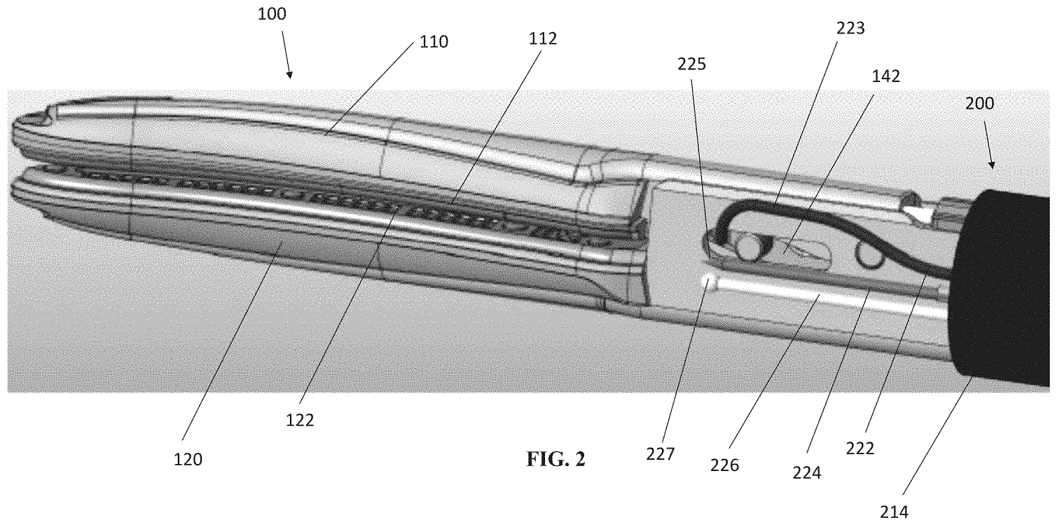

Handle assembly 30 includes movable handle 40 and a fixed handle 50. Fixed handle 50 is integrally associated with housing 20 and movable handle 40 is movable relative to fixed handle 50 between an initial position, wherein movable handle 40 is spaced-apart from fixed handle 50, and a compressed position, wherein movable handle 40 is compressed towards fixed handle 50. Drive assembly 140 includes a drive bar 142 (FIG. 2) that is slidably disposed within shaft assembly 80 and configured to operably couple movable handle 40 with end effector assembly 100 such that movement of movable handle 40 between the initial position and the compressed position moves one or both of jaw members 110, 120 between the spaced-apart position and the approximated position.

As noted above, bipolar activation switch 172 of bipolar activation assembly 170 is provided to selectively supply energy to surfaces 112, 122 of jaw members 110, 120, respectively, of end effector assembly 100. Upon sufficient compression of movable handle 40 relative to fixed handle 50, a portion of movable handle 40 is urged into contact with bipolar activation assembly 170 so as to activate bipolar activation switch 172.

Trigger 62 of trigger assembly 60 is operably coupled with the knife of knife assembly 160 and is selectively actuatable relative to housing 20 from an un-actuated position to an actuated position to translate the knife relative to jaw members 110, 120 from a retracted position wherein the knife is disposed proximally of jaw members 110, 120, to an extended position, wherein the knife extends at least partially between jaw members 110, 120 and through the knife channel(s) (not shown) thereof to cut tissue grasped between jaw members 110, 120.

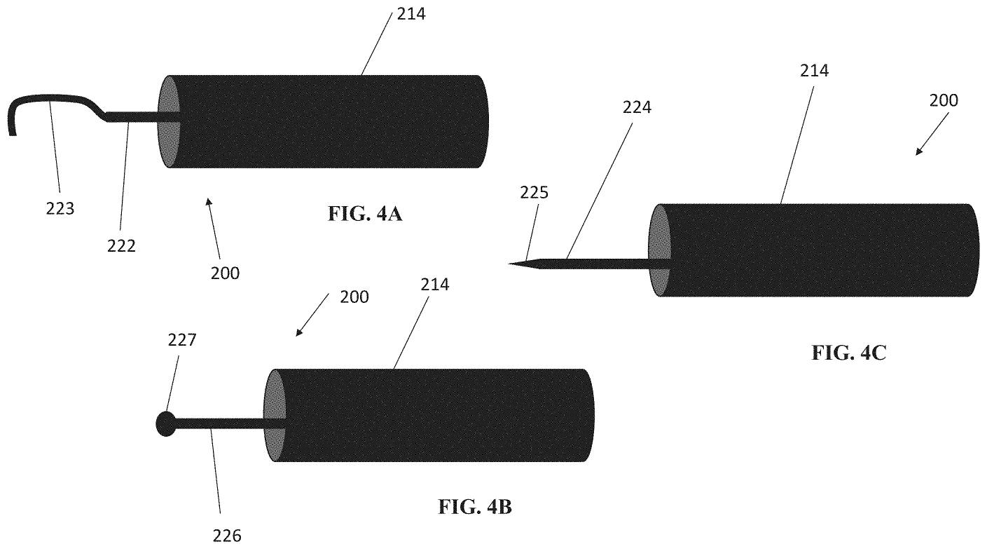

With additional reference to FIGS. 4A-4C, monopolar assembly 200 includes a sheath assembly 210 and a plurality of monopolar electrodes 222, 224, 226. Sheath assembly 210 includes an elongated insulative sheath 214 that is slidably relative to shaft assembly 80 and end effector assembly 100 between a storage position (FIG. 2), wherein elongated insulative sheath 214 is disposed proximally of end effector assembly 100, and a use position (FIGS. 4A-4C), wherein elongated insulative sheath 214 is substantially disposed about end effector assembly 100.

Monopolar electrodes 222, 224, 226 of monopolar assembly 200 include different configurations to facilitate different tissue treatments, treating different tissue types, and/or for other purposes. More specifically, monopolar electrode 222 defines a hooked distal end portion 223, monopolar electrode 224 defines a conical distal end portion 225, and monopolar electrode 226 defines a ball-shaped distal end portion 227. However, other suitable distal end configurations for use with monopolar electrodes 222, 224, 226 are also contemplated such as, for example, a loop electrode, a spatula electrode, a U-shaped electrode, etc. Further, as an alternative to monopolar electrodes 222, 224, 226, one or more of the monopolar electrodes 222, 224, 226 may be replaced with another energy-delivery device (e.g., a bipolar, microwave, thermal, light-energy, ultrasonic or other suitable energy-providing probe); a mechanical device (e.g., a grasper, a knife, a sharp implement, a saw, etc.); and/or any other suitable surgical tool (e.g., a surgical camera, a sensor (for sensing temperature, electrical properties, mechanical properties, etc.), a light source, etc. Selection assembly 400, as detailed below, enables the selection of one of monopolar electrodes 222, 224, 226 for mechanical and electrical coupling with deployment and retraction mechanism 300 and both the source of energy (not shown) and monopolar activation assembly 180, respectively, to enable the deployment and activation of the selected monopolar electrode 222, 224, 226 for use in the monopolar mode of operation.

The selected monopolar electrode 222, 224, 226 is configured for movement, together with the movement of elongated insulative sheath 214, between a storage position (FIG. 2), wherein the selected monopolar electrode 222, 224, 226 is retracted relative to jaw members 110, 120 of end effector assembly 100, and a use position (FIGS. 4A-4C), wherein the selected monopolar electrode 222, 224, 226 extends distally from end effector assembly 100 to facilitate treating tissue therewith. Further, in the use position (FIGS. 4A-4C), energy may be supplied to wherein the selected monopolar electrode 222, 224, 226, e.g., via activation of either of the activation switches 182 of monopolar activation assembly 180, for treating tissue in the monopolar mode of operation. The non-selected monopolar electrodes 222, 224, 226 remain in the storage position, un-energizable, regardless of the position and/or activation state of the selected monopolar electrode 222, 224, 226.

Rotating assembly 70 is rotatably disposed but longitudinally constrained within a vertically-oriented slot 26 defined within housing 20. Rotating assembly 70 extends at least partially through slot 26 on either side of housing 20 to enable manipulation of rotating assembly 70 on either exterior side of housing 20 to thereby rotate shaft assembly 80, end effector assembly 100, knife assembly 160, and monopolar assembly 200 in cooperation with one another relative to housing 20.

Referring back to FIGS. 1-3, deployment and retraction mechanism 300 is configured for selectively transitioning monopolar assembly 200 between its storage condition and its use condition, although deployment and retraction mechanism 300 may similarly be used in connection with any suitable surgical instrument for deploying and retracting any suitable deployable component(s). Deployment and retraction mechanism 300 generally includes a gear box 302 mounted within housing 20, a gear assembly 330 operably disposed within gear box 302, a pair of rotatable actuators 380 operably coupled to the input of gear assembly 330, and an output driver 394 operably engaged with elongated insulative sheath 214 of monopolar assembly 200 and configured to operably engage the selected monopolar electrode 222, 224, 226 of monopolar assembly 200 to thereby operably couple elongated insulative sheath 214 and the selected monopolar electrode 222, 224, 226 with the output of gear assembly 330. More specifically, a suitable connector (not shown), e.g., a linkage, sleeve, bar, combinations thereof, etc., extends between output driver 394 and elongated insulative sheath 214 to engage output driver 394 and elongated insulative sheath 214 with one another such that output driver 394 and elongated insulative sheath 214 move together with one another. As detailed below, selection assembly operably engages output driver 394 with the selected monopolar electrode 222, 224, 226 such that output driver 394 and the selected monopolar electrode 222, 224, 226 move together with one another (and with elongated insulative sheath 214).

Deployment and retraction mechanism 300 is configured to enable both deployment and retraction of monopolar assembly 200 in a push-push manner, e.g., wherein monopolar assembly 200 is both deployed and retracted by pushing either of rotatable actuators 380 in the same direction, return monopolar assembly 200 back to its previous condition in the event of an incomplete actuation, retain monopolar assembly 200 in the use condition or the storage condition upon a full actuation, provide an advantageous gear ratio for deploying and retracting monopolar assembly 200, actuate movable handle 40 to approximate jaw members 110, 120 prior to deployment of monopolar assembly 200 if necessary, permit the supply of energy to monopolar assembly 200 only when monopolar assembly 200 is disposed in the use condition, and permit the supply of energy to jaw members 110, 120 only when monopolar assembly 200 is disposed in the storage condition. Deployment and retraction mechanism 300 is described in greater detailed in U.S. Pat. No. 10,039,593, the entire contents of which are hereby incorporated herein by reference. However, other suitable deployment and retraction mechanisms 300 are also contemplated. Further details of and/or additional features configured for use with surgical instrument 10 can be found in U.S. Pat. No. 10,039,593, and/or in U.S. Pat. No. 10,537,381, the entire contents of which are hereby incorporated herein by reference.

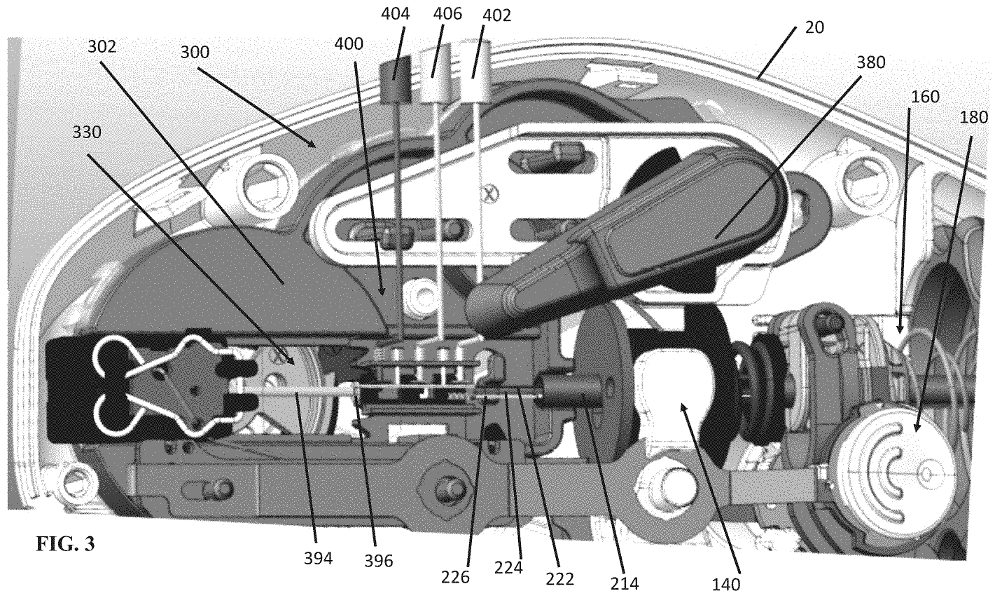

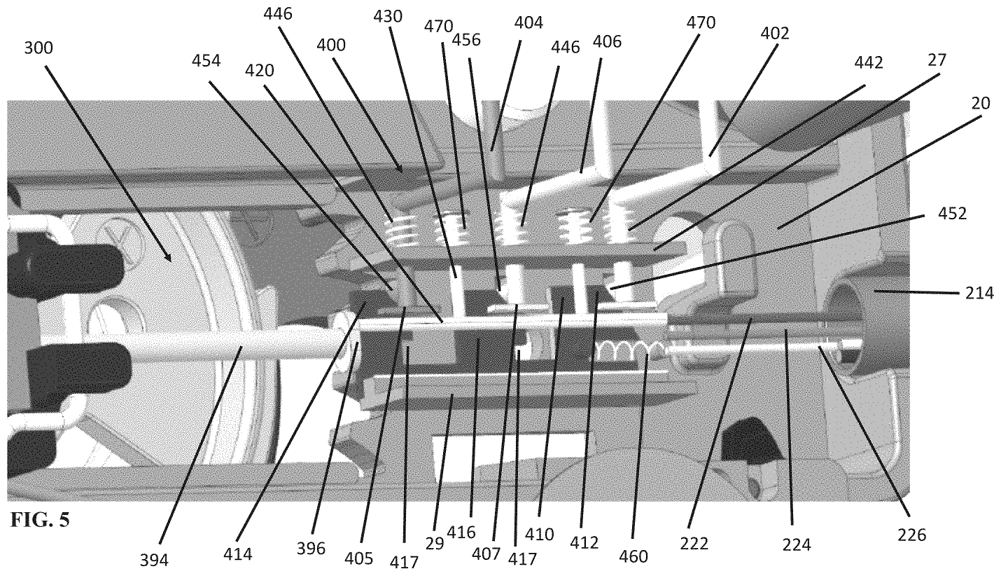

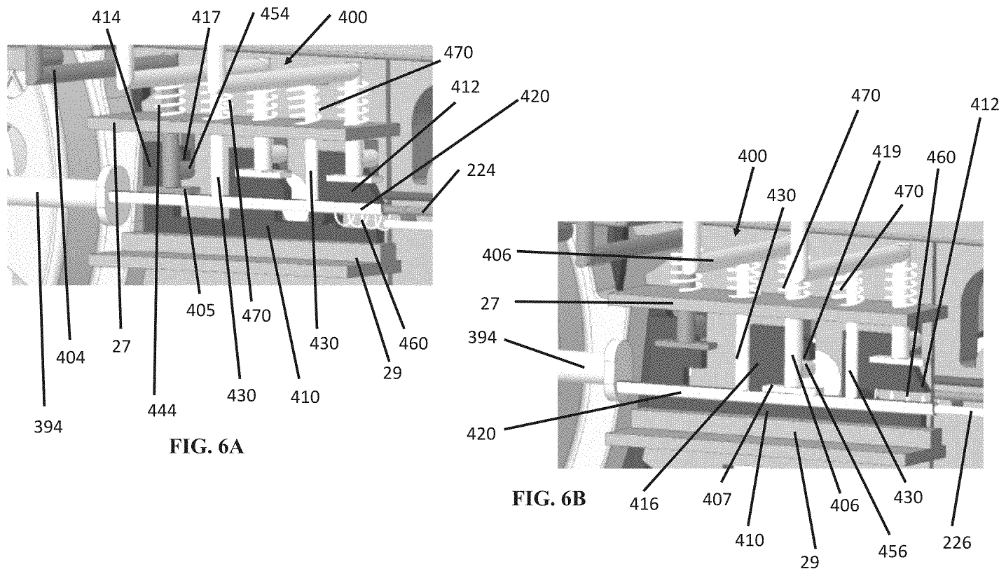

Turning to FIGS. 5, 6A, and 6B, in conjunction with FIGS. 1 and 3, as noted above, selection assembly 400 enables the selection of one of monopolar electrodes 222, 224, 226 for mechanical and electrical coupling with deployment and retraction mechanism 300 and both the source of energy (not shown) and monopolar activation assembly 180 (FIG. 3), respectively, to enable the deployment and activation of the selected monopolar electrode 222, 224, 226 for use in the monopolar mode of operation. Selection assembly 400 is mounted within housing 20 between upper and lower support shelves 27, 29 disposed within housing 20. Selection assembly 400 includes first, second, and third selectors 402, 404, 406 protruding from housing 20 to enable manual depression of one of the selectors 402, 404, 406 by a user; second and third drivers 405, 407 connected to the second and third selectors 404, 406, respectively; a sliding frame 410 including first, second, and third cam wedges 412, 414, 416, respectively, operably associated with respective first, second, and third selectors 402, 404, 406; and a coupling shaft 420 supported by one or more support posts 430.

Selectors 402, 404, 406 extend from the manually depressible portions thereof, externally of housing 20, into housing and through apertures defined within upper support shelf 27. Biasing members 442, 444, 446 such as, for example, coil springs, are disposed about selectors 402, 404, 406, respectively, and are seated on upper support shelf 27 to bias the manually depressible portions of selectors 402, 404, 406 outwardly from housing 20 towards un-actuated positions. Second and third drivers 405, 407 are disposed at the free ends of second and third selectors 404, 406, respectively, opposite the respective manually depressible portions thereof. First, second, and third selectors 402, 404, 406 further include respective cam bosses 452, 454, 456 extending transversely therefrom.

Sliding frame 410 is slidingly supported between upper and lower support shelves 27, 29 of housing 20 and is permitted to slid longitudinally within housing 20 between upper and lower support shelves 27, 29. A biasing member 460, e.g., a coil spring, biases sliding frame 410 distally. As noted above, sliding frame 410 includes first, second, and third cam wedges 412, 414, 416; second and third cam wedges 414, 416 define catch basins 415, 417, respectively.

Coupling shaft 420, as noted above, is supported by one or more support posts 430 slidably extending through apertures defined within upper support shelf 27 to enable vertical sliding of coupling shaft 420 between a first position (FIG. 5), a second position (FIG. 6A), and a third position (FIG. 6B). A biasing member 470 such as, for example, a coil spring, is disposed about each coupling shaft 420 and is seated on upper support shelf 27 to bias coupling shaft 420 towards the first position (FIG. 5). Coupling shaft 420 is vertically supported by support posts 430, as noted above, but is longitudinally slidable relative to support posts 430 via, for example, engagement of the free ends of support posts 430 within a slide track (not explicitly shown) defined within coupling shaft 420. The slide track and free ends of support posts 430 may define complementary configurations such as, for example, T-slot and T-shape configurations.

Continuing with reference to FIGS. 5, 6A, and 6B, in conjunction with FIGS. 1 and 3, the proximal end of coupling shaft 420 is configured to be driven longitudinally by output driver 394 via output driver 394 contacting and urging coupling shaft 420 distally in response to actuation of output driver 394. The driving end 396 of output driver 394 is configured such that output driver 394 is capable of contacting and urging coupling shaft 420 longitudinally regardless of the vertical position of coupling shaft 420, e.g., regardless of whether coupling shaft 420 is disposed in the first position (FIG. 5), the second position (FIG. 6A), or the third position (FIG. 6B). The distal end of coupling shaft 420 is configured, in response to longitudinal urging by output driver 394, to drive translation of a corresponding one of the monopolar electrodes 222, 224, 226 (or drivers thereof) depending upon the position of coupling shaft 420. That is, with coupling shaft 420 disposed in the first position (FIG. 5), longitudinal urging by output driver 394 translates coupling shaft 420 to thereby translate monopolar electrode 222 (or the driver thereof) between the storage and use position thereof with coupling shaft 420 disposed in the second position (FIG. 6A), longitudinal urging by output driver 394 translates coupling shaft 420 to thereby translate monopolar electrode 224 (or the driver thereof) between the storage and use position thereof; and with coupling shaft 420 disposed in the third position (FIG. 6B), longitudinal urging by output driver 394 translates coupling shaft 420 to thereby translate monopolar electrode 226 (or the driver thereof) between the storage and user position thereof. Monopolar electrodes 222, 224, 226 may be biased towards the storage positions via suitable biasing members (not shown) such that, upon return of coupling shaft 420 proximally, the deployed monopolar electrode 222, 224, 226 is likewise returned proximally to the storage position thereof. Alternatively, coupling shaft 420 may be configured to releasably engage the adjacent monopolar electrode 222, 224, 226 (depending upon the position thereof) to bi-directionally translate the adjacent monopolar electrode 222, 224, 226.

As detailed above, elongated insulative sheath 214 is also operably engaged with output driver 394 such that the selected monopolar electrode 222, 224, 226 and elongated insulative sheath 214 move together with one another between the storage and use positions thereof regardless of the position of coupling shaft 420 and, thus, regardless of which monopolar electrode 222, 224, 226 is selected for deployment.

Selection of one of monopolar electrodes 222, 224, 226 using selection assembly 400, as noted above, is achieved by manually depressing the corresponding selector 402, 404, 406. Further, selection assembly 400, is initially disposed, e.g., in the absence of depression of one of selectors 404, 406, in a first condition, corresponding to the first position of coupling shaft 420 (FIG. 5), wherein the first monopolar electrode 222 is selected. Further, if selection assembly 400 is disposed in a second or third condition, corresponding to the second or third position (FIGS. 6A and 6B) of coupling shaft 420, respectively, wherein the respective second or third monopolar electrode 224, 226 is selected, depression of selector 402 will return selection assembly 400 to the first condition, as detailed below.

With particular reference to FIG. 6A, in order to select monopolar electrode 224, selector 404 is depressed. Depression of selector 404 urges driver 405 towards and into contact with coupling shaft 420 such that driver 405 urges coupling shaft 420 from the first position (FIG. 5) to the second position (FIG. 6A). As selector 404 is depressed such that driver 405 moves coupling shaft 420 in this manner, cam boss 454 is urged into contact with cam wedge 414 of sliding frame 410 to thereby urge sliding frame 410 proximally as selector 404 is depressed. When selector 404 reaches the fully depressed position, corresponding to the position wherein driver 405 has moved coupling shaft 420 to the second position, cam boss 454 slides off cam wedge 414 of sliding frame 410 allowing sliding frame 410 to return distally under the bias of biasing member 460. As a result, cam boss 454 is received within catch basins 415, thereby retaining selector 404 in the depressed position such that driver 405 retains coupling shaft 420 in the second position. In this second position, as noted above, longitudinal urging by output driver 394, e.g., in response to actuation of deployment and retraction assembly 300, translates coupling shaft 420 to thereby translate monopolar electrode 224 (or the driver thereof) between the storage and use position thereof (or to the use position while a return force returns monopolar electrode 224 to the storage position thereof upon retraction of output driver 394).

With particular reference to FIG. 6B, in order to select monopolar electrode 226, selector 406 is depressed. Depression of selector 406 urges driver 407 towards and into contact with coupling shaft 420 such that driver 475 urges coupling shaft 420 to the third position (FIG. 6B). As selector 406 is depressed such that driver 407 moves coupling shaft 420 in this manner, cam boss 456 is urged into contact with cam wedge 416 of sliding frame 410 to thereby urge sliding frame 410 proximally as selector 406 is depressed. When selector 406 reaches the fully depressed position, corresponding to the position wherein driver 407 has moved coupling shaft 420 to the third position, cam boss 456 slides off cam wedge 416 of sliding frame 410 allowing sliding frame 410 to return distally under the bias of biasing member 460. As a result, cam boss 456 is received within catch basins 417, thereby retaining selector 406 in the depressed position such that driver 407 retains coupling shaft 420 in the third position. In this third position, as noted above, longitudinal urging by output driver 394, e.g., in response to actuation of deployment and retraction assembly 300, translates coupling shaft 420 to thereby translate monopolar electrode 226 (or the driver thereof) between the storage and use position thereof (or to the use position while a return force returns monopolar electrode 226 to the storage position thereof upon retraction of output driver 394).

Referring to FIGS. 5, 6A, and 6B, in order to return selection assembly 400 to the first position, selector 402 is depressed. Depression of selector 402 moves selector 402 and, thus, moves cam boss 452 into contact with cam wedge 412 of sliding frame 410 to thereby urge sliding frame 410 proximally as selector 402 is depressed. Sliding frame 410 is moved sufficiently proximally such that cam boss 454 of selector 404 is displaced from catch basins 415 and/or such that cam boss 456 of selector 406 is displaced from catch basins 417. As a result, coupling shaft 420 is allowed to return vertically under the bias of biasing members 470 to the first position. In this first position, as noted above, longitudinal urging by output driver 394, e.g., in response to actuation of deployment and retraction assembly 300, translates coupling shaft 420 to thereby translate monopolar electrode 222 (or the driver thereof) between the storage and use position thereof (or to the use position while a return force returns monopolar electrode 222 to the storage position thereof upon retraction of output driver 394).

Turning back to FIGS. 3 and 5, with respect to electrically coupling the selected monopolar electrode 222, 224, 226 with the source of energy (not shown) and monopolar activation assembly 180, output driver 394, or at least driving portion 396 thereof, is formed at least partially from an electrically-conductive material and/or includes electrical connectors disposed thereon or therein. The electrically-conductive portions and/or electrical connectors of output driver 394 are connected, directly or indirectly, to the source of energy (not shown) and monopolar activation assembly 180. Coupling shaft 420, in turn, is formed at least partially from an electrically-conductive material and/or includes electrical connectors disposed thereon or therein such that, regardless of the position of coupling shaft 420, coupling shaft 420 is electrically coupled to output driver 394 when output driver 394 is disposed in a deployed position. When coupling shaft 420 is moved, via output driver 394, to a deployed position to contact a corresponding one of the monopolar electrodes 222, 224, 226 to deploy the monopolar electrodes 222, 224, 226, electrical communication is established between the electrically-conductive portions and/or electrical connectors of coupling shaft 420 and the selected, deployed monopolar electrode 222, 224, 226. Thus, upon activation, monopolar energy can be delivered to (and only to) the selected, deployed monopolar electrode 222, 224, 226. In this manner, a safety feature is provided wherein energy is only capable of being delivered to the selected, deployed monopolar electrode 222, 224, 226.

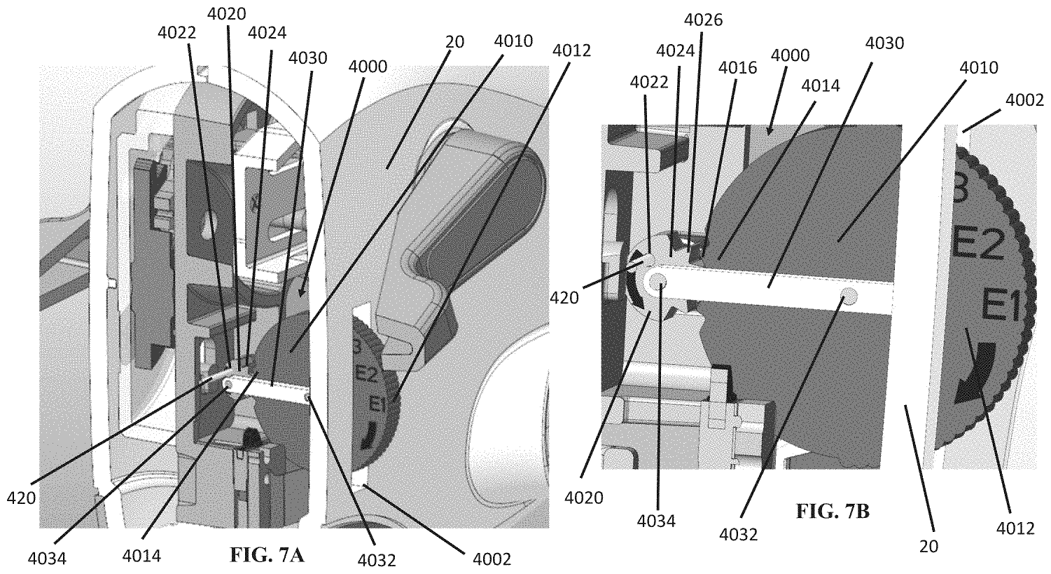

Referring to FIGS. 7A and 7B, in conjunction with FIGS. 3 and 5, another selection assembly provided in accordance with the present disclosure and configured for use with surgical instrument 10 (FIG. 1) or any other suitable surgical instrument is shown generally identified by reference numeral 4000. Selection assembly 4000 is similar to selection assembly 400 (FIGS. 5-6B) in that selection assembly enables the selective movement of coupling shaft 420 between first, second, and third positions to thereby operably couple output driver 394 to the first, second, or third monopolar electrode 222, 224, 226, respectively, to enable selective deployment and retraction thereof. However, the manner in which selection assembly 4000 effects movement of coupling shaft 420 to and retention of coupling shaft 420 in the selected first, second, or third position is different from that of selection assembly 400 (FIGS. 5-6B). Thus, these differences are described in detail below while similarities are summarily described or omitted entirely.

Selection assembly 4000 includes an input gear 4010, an output gear 4020, a support frame 4030, and one or more biasing members (not explicitly shown). Input gear 4010 is rotatably supported by frame 4030 and, thus, relative to housing 20, via a pin 4032. A manipulation portion 4012 of input gear 4010 extends outwardly through a slot 4002 defined within housing 20 of instrument 10 to enable manual rotation of input gear 4010 about pin 4032 and relative to housing 20. Manipulation portion 4012 may further include indicia indicating the current selection of selection assembly 4000, other selection of selection assembly 4000, and/or a directional indicator indicating the rotation direction to transition between the current selection and another selection of selection assembly 4000. Input gear 4010 further includes a gear portion 4014 defining a plurality of gear teeth 4016.

Output gear 4020 of selection assembly 4000 is rotatably supported by frame 4030 and, thus, relative to housing 20, via a pin 4034. Output gear 4020 includes an aperture 4022 including coupling shaft 420 slidably received therethrough. Aperture 4022 is offset from the center of output gear 4020, e.g., offset from pin 4034, such that aperture 4022 and, thus, coupling shaft 420 are translated along an arc in response to rotation of output gear 4020 between first, second, and third positions corresponding to selection of the first, second, and third monopolar electrode 222, 224, 226, respectively. Output gear 4020 further includes a gear portion 4024 defining a plurality of gear teeth 4026 disposed in meshed engagement with gear teeth 4016 of input gear 4010 such that rotation of input gear 4010 in a first direction rotates output gear 4020 in a second, opposite direction. Output gear 4020, in aspects, defines a minor gear relative to input gear 4010 which defines the major gear, although other configurations are also contemplated.

The one or more biasing members are provided to maintain selection assembly 4000 in the selected position. That is, the one or more biasing members bias selection assembly 4000 towards its current a stationary position such that selection assembly 4000 is maintained in the selected position in the absence of sufficient urging of input gear 4010 to overcome the bias and rotate input gear 4010 to another selected position, at which point the one or more biasing members bias selection assembly 4000 toward the new selected position.

In use, input gear 4010 is rotated to a desired position, e.g., corresponding to selection of one of the first, second, or third monopolar electrode 222, 224, 226. Upon such rotation, coupling shaft 420 is thus moved to a corresponding first, second, or third position to thereby operably couple output driver 394 to the first, second, or third monopolar electrode 222, 224, 226, respectively, to enable selective deployment and retraction thereof. Upon deployment and retraction, coupling shaft 420 slides longitudinally through aperture 4023 and relative to output gear 4020.

While several configurations of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical instrument, comprising:
a housing;
at least three deployable components, each deployable component of the at least three deployable components selectively deployable relative to the housing from a storage position to a use position;
a deployment and retraction assembly including an actuator, an output driver, and a gear assembly operably coupled between the actuator and the output driver such that actuation of the actuator drives the output driver; and
a selection assembly operably coupled to the deployment and retraction assembly, the selection assembly including a coupling shaft movable between at least a first position wherein the coupling shaft is operably coupled between the output driver and a first deployable component of the at least three deployable components such that actuation of the actuator drives the output driver to deploy the first deployable component, a second position wherein the coupling shaft is operably coupled between the output driver and a second deployable component of the at least three deployable components such that actuation of the actuator drives the output driver to deploy the second deployable component, and a third position wherein the coupling shaft is operably coupled between the output driver and a third deployable component of the at least three deployable components such that actuation of the actuator drives the output driver to deploy the third deployable component.

2. The surgical instrument according to claim 1, wherein the output driver is adapted to connect to a source of energy, and wherein the coupling shaft is configured to electrically couple the output driver to the first, second, and third deployable components in the respective first, second, and third positions of the coupling shaft.

3. The surgical instrument according to claim 2, wherein at least one of the first, second, or third deployable components is a monopolar electrode configured to supply monopolar energy to tissue in the use position thereof.

4. The surgical instrument according to claim 1, wherein the selection assembly includes at least second and third selectors extending from the housing, each of the second and third selectors including a driver at a free end thereof, wherein depression of the second and third selectors moves the coupling shaft, under urging by the corresponding driver, to the second and third positions, respectively.

5. The surgical instrument according to claim 4, wherein the selection assembly includes a first selector extending from the housing, wherein depression of the first selector releases the coupling shaft for return from the second or third position thereof to the first position thereof.

6. The surgical instrument according to claim 1, wherein the selection assembly includes an input gear extending from the housing and an output gear coupled to the input within the housing, the output gear operably coupled to the coupling shaft such that rotation of the input gear to first, second, and third orientations, respectively, moves the coupling shaft to the first, second, and third positions, respectively.

7. The surgical instrument according to claim 1, wherein rotatable actuation of the actuator of the deployment and retraction assembly drives the output driver of the deployment and retraction assembly to translate longitudinally.

8. The surgical instrument according to claim 7, wherein a first rotational actuation of the actuator in a first rotational direction translationally drives the output driver in a first translational direction, and wherein a second, subsequent rotational actuation of the actuator in the first rotational direction translationally drives the output driver in second, opposite translational direction.

9. The surgical instrument according to claim 1, wherein the coupling shaft moves vertically between the first, second, and third positions.

10. The surgical instrument according to claim 1, wherein the coupling shaft moves along an arc between the first, second, and third positions.

11. A surgical instrument, comprising:
a housing;
a shaft assembly extending distally from the housing;
an end effector assembly disposed at a distal end of the shaft assembly;
a plurality of deployable components, each deployable component of the plurality of deployable components selectively deployable relative to the end effector assembly from a storage position to a use position;
a deployment and retraction assembly including an actuator, an output driver, and a gear assembly operably coupled between the actuator and the output driver such that actuation of the actuator in a first manner drives the output driver in a second, different manner; and
a selection assembly operably coupled to the deployment and retraction assembly, the selection assembly transitionable between a plurality of different configurations to thereby operably couple the output driver of the deployment and retraction assembly to a different deployable component of the plurality of deployable components in each of the plurality of different configurations of the selection assembly,
wherein actuation of the actuator drives the output driver to deploy the deployable component of the plurality of deployable components that is operably coupled to the output driver as selected based on the configuration of the selection assembly.

12. The surgical instrument according to claim 11, wherein the selection assembly includes a plurality of selectors extending from the housing, wherein depression of each selector of the plurality of selectors transitions the selection assembly to a different configuration of the plurality of different configurations.

13. The surgical instrument according to claim 11, wherein the selection assembly includes an input gear extending from the housing and rotatable between a plurality of different input gear orientations, wherein rotation of the input gear to each orientation of the different input gear orientations transitions the selection assembly to a different configuration of the plurality of different configurations.

14. The surgical instrument according to claim 11, wherein the first manner of actuation of the actuator of the deployment and retraction assembly is a rotational actuation and wherein the second manner of actuation of the output driver of the deployment and retraction assembly is a translational driving.

15. The surgical instrument according to claim 14, wherein a first rotational actuation of the actuator in a first rotational direction translationally drives the output driver in a first translational direction, and wherein a second, subsequent rotational actuation of the actuator in the first rotational direction translationally drives the output driver in second, opposite translational direction.

16. The surgical instrument according to claim 11, wherein the selection assembly includes a coupling shaft operably coupled with the output driver, the coupling shaft configured to move between a plurality of different positions, each position of the plurality of different positions corresponding to one of the different configurations of the selection assembly, wherein in each position of the plurality of different positions, the coupling shaft is operably coupled to a different deployable component of the plurality of deployable components.

17. The surgical instrument according to claim 16, wherein the coupling shaft is movable in a first direction between the plurality of different positions and is movable in a second, perpendicular direction upon driving of the output driver to thereby deploy the corresponding deployable component of the plurality of deployable components.

18. The surgical instrument according to claim 17, wherein the selection assembly includes a plurality of selectors extending from the housing, wherein depression of each selector of the plurality of selectors moves the coupling shaft to a different position of the plurality of different positions.

19. The surgical instrument according to claim 16, wherein the coupling shaft is movable along an arc between the plurality of different positions and is movable in a second direction perpendicular to a plane containing the arc upon driving of the output driver to thereby deploy the corresponding deployable component of the plurality of deployable components.

20. The surgical instrument according to claim 19, wherein the selection assembly includes a selection gear extending from the housing, wherein rotation of the selection gear moves the coupling shaft along the arc between the plurality of different positions.
